(19) United States
(12) Patent Application Publication
(10) Pub. No.: US 2024/0261501 A1
(43) Pub. Date: Aug. 8, 2024

(54) DUAL MODE AND HYBRID PUMP AND CONTAINER SYSTEMS

(71) Applicant: ICU Medical, Inc., San Clemente, CA (US)

(72) Inventors: James Duane JACOBSON, Lindenhurst, IL (US); Robert L. HLINSKY, Glenview, IL (US)

(21) Appl. No.: 18/681,018

(22) PCT Filed: Dec. 16, 2022
(86) PCT No.: PCT/US2022/081881

§ 371 Date: Feb. 2, 2024

Related U.S. Application Data
(60) Provisional application No. 63/265,901, filed on Dec. 22, 2021.

(51) Int. Cl. A61M 5/172; A61M 5/14; A61M 5/142

(52) U.S. Cl. CPC A61M 5/172; A61M 5/1413; A61M 5/1415; A61M 5/14244; A61M 2202/048; A61M 2205/3303; A61M 2205/505; A61M 2230/40

(57) ABSTRACT

A multi-function, modular pump system can have a portable infusion pump configured for ambulatory use. The portable infusion pump can have a battery, a portable user interface, and a docking module or controller/container. The docking module or controller/container can have a security feature configured to prevent removal or unauthorized access and a pump mount configured to engage and stabilize the pump within the container, allow pump to maintain infusion continuity when removed, and enable rapid disengagement when authorized. An external user interlace can allow for control of the contained pump, review of pump run status, log event history, settings, and configurations. A machine interface can allow control signals to pass from external interface to a contained, engaged portable pump. A power module can be configured to provide power to both the contained portable infusion pump and the pump docking module or controller/container. A simplified extending patient control can be configured to provide a bolus dose. The portable pump can be configurable for use in two modes: portable mode, and secured mode.

### INCORPORATION BY REFERENCE TO PRIORITY APPLICATION

This application is based upon and claims the benefit of priority from U.S. Provisional Patent Application No. 63/265,901, filed on Dec. 22, 2021. Moreover, any and all applications for which a foreign or domestic priority claim is identified in the Application Data Sheet as filed with the present application are hereby incorporated by reference under 37 CFR § 1.57. The entire contents of each of the above-listed items is hereby incorporated into this document by reference and made a part of this specification for all purposes, for all that each contains.

### BACKGROUND

This disclosure relates to parenteral infusion pumps, including electronically controlled parenteral infusion pumps, configured for both ambulatory and bedside use.

### RELATED ART

Infusion pumps are often designed for either bedside use or for ambulatory use. There is a need for systems that allow secure use in multiple settings, including for both immobile and mobile patients.

### SUMMARY

An ambulatory pump may, independently, not have sufficient features or security to allow its use with controlled substances in acute care or alternate care environments, per guidelines established for preventing diversion of controlled substances. On the other hand, sophisticated bedside pumps may be bulky and include features that are not needed for a typical ambulatory patient. The present disclosure provides systems and methods for addressing these and related problems.

For example, a multi-function, modular pump system can have a portable infusion pump. The pump can be independently operational and configured for ambulatory use, having: a battery (e.g., a rechargeable battery) and a power input structure connected thereto; a portable user interface; a pump controller; and a separate pump controller/container configured for preventing diversion of controlled substances. Example controller/containers are also described herein as an “accessory” to a pump, and as a pump docking container. Such an accessory or container can be larger than the pump itself and therefore configured less for ambulatory use and more for use in a bedside (e.g., critical care) environment. The controller/docking container can have: a security feature configured to prevent removal or unauthorized access to and/or removal of the portable pump and medication (e.g., pole-mounted lock box and/or additional features configured to prevent unauthorized removal of the controller/docking container and pump from the IV pole stand); a pump mount configured to engage and stabilize the pump within the container, allowing a pump to maintain infusion continuity when removed, and enable rapid disengagement when authorized: an external user interface allowing for control of the contained pump (e.g., large touch screen) and review of pump run status, log event history (such as timing of patient boluses requested and delivered via patient bolus button and total medication amount delivered by hour), settings, configurations, etc.; a machine interface allowing control signals to pass from external interface to contained, engaged portable pump (e.g., docking station, NFC chip(s), transceiver(s), etc.); and a power module configured to provide power to both the contained portable infusion pump and the pump controller/docking container. The system can also have a simplified extending patient control configured to provide a bolus dose and/or provide infusion status to the patient. The portable pump in such a system can be configurable for use in two modes: (1) portable mode, where it operates independently of the pump controller/docking container; and (2) secured mode, wherein it fits within and cooperates with the pump controller/docking container to provide infusion under restricted control and access (restricting control and access applies to patients, clinicians, family members, etc). In secured mode the pump may also enjoy additional functionality that could be therefore left out of the self-contained portable pump such as Wi-Fi communications, cellular communications, interface/support for sensor or monitoring capabilities like SpO2, EtCO2, minute ventilation, patient vital signs, etc. to monitor for respiratory depression.

In some embodiments, a multi-function, modular pump system can comprise a stationary docking station and a portable pump. The docking station can have at least one security feature configured to prevent unauthorized removal of or access to the docking station, the portable pump (which can be attachable to the docking station), and a medication reservoir container (which can be configurable to be in fluid communication with the pump). The station can have a pump mount configured to engage and stabilize the pump within the docking station, allow the pump to maintain infusion continuity when removed, and enable rapid, non-technical disengagement of the pump from the docking station (e.g., when authorized). The station can have a stationary external user interface configured to allow user control of the portable pump and review of pump run status, event history, and settings, for example. The system can comprise the portable pump, which can comprise an infusion pump configurable to be separable from the docking station and, when separated, function independently for ambulatory use. The portable pump can further comprise: a battery and a power input structure connected to the battery; a portable user interface; and a medication reservoir container.

The stationary docking station in the systems described above can further comprise a machine interface configured to allow control signals and other information to pass between the external user interface and the contained, engaged portable pump. A power module can be configured to provide power to both the contained portable infusion pump and the stationary docking station. The external user interface can comprise a large touchscreen. The battery of the portable infusion pump can be rechargeable, and the power input structure can be configured to interface with the power module and a charging structure in the stationary docking station to recharge the battery. The system can further comprise a simplified extending patient control configured to provide a bolus dose. The portable infusion pump can be configurable for use in portable mode and secured mode. In some embodiments of portable mode, the pump can operate independently of the stationary docking station. In some embodiments of secured mode, the pump can fit within and cooperate with the stationary docking station, and the system can support additional functionality comprising one or more of stronger communications hardware and sensor support. Stronger communications hardware can comprise Wi-Fi communications or cellular communications, and stronger sensor support can comprise interfaces and support monitoring information including one or more of SpO2, EtCO2, minute ventilation, and patient vitals. Sensor support can be configured to monitor for opioid induced respiratory depression.

In some embodiments, a multi-function, modular pump system can comprise a portable infusion pump configurable to function independently for ambulatory use. The pump can include: a battery and a power input structure connected to the battery; a portable user interface; a medication reservoir container; and a pump docking container. The container can include: at least one security feature configured to prevent unauthorized removal of or access to the docking container, the portable pump and the medication reservoir container. The container can further include a pump mount configured to engage and stabilize the pump within the container, allow the pump to maintain infusion continuity when removed, and enable rapid disengagement when authorized. An external user interface can be configured to allow user control of the contained portable infusion pump and review of pump run status, event history, and settings.

The pump docking container in the system described above can further comprise a machine interface configured to allow control signals and other information to pass between the external user interface and the contained, engaged portable pump. A power module can be configured to provide power to both the contained portable infusion pump and the pump docking container. The external user interface can comprise a large touch-screen. The battery of the portable infusion pump can be rechargeable, and the power input structure can be configured to interface with the power module and a charging structure in the pump docking container to recharge the battery. The system can further comprise a simplified extending patient control configured to provide a bolus dose. The portable infusion pump can be configurable for use in portable mode and secured mode. In some embodiments of portable mode, the pump can operate independently of the pump docking container. In some embodiments of secured mode, the pump can fit within and cooperate with the pump docking container, and the system can support additional functionality comprising one or more of stronger communications hardware and sensor support. Stronger communications hardware can comprise Wi-Fi communications or cellular communications, and stronger sensor support can comprise interfaces and support monitoring information including one or more of SpO2, EtCO2, minute ventilation, and patient vitals. Sensor support can be configured to monitor for opioid induced respiratory depression.

In some embodiments, a modular pump system can comprise: a portable infusion pump configurable for independent operation in a first mode and having a medication container incorporated therewith; and a pump docking module configured to securely house the portable infusion pump and medication container, the docking module comprising a user interface and configured to operatively and persistently communicate with the portable infusion pump while the pump is housed therein.

In the system described above, the pump docking module can be configured to engage and stabilize the pump therein, allowing pump to maintain infusion continuity when removed, and enable rapid disengagement when authorized. The pump docking module user interface can comprise a color touch screen that provides interactive control and display responsibilities of the portable infusion pump when that pump is mounted within the docking module. The docking module user interface can be configured to assume interactive control and display responsibilities of the portable infusion pump when that pump is mounted within the docking module. In some embodiments, the docking module locks to secure the pump and medication within the module and can be unlocked by one or more of: a physical key, a passcode entry, and a clinician badge. In some embodiments, the docking module is secured to IV pole such that it is releasable only by one or more of a physical key, a passcode entry, and a clinician badge. In some embodiments, a power submodule within the docking module is configured to convert AC power, power the docking module features, and power and recharge a battery of the portable infusion pump. In some embodiments, the system further comprises a patient bolus cord configured to communicate to the pump directly or through the docking module. In some embodiments, a patient bolus control wirelessly communicates to the pump and includes a battery rechargeable by the docking module. In some embodiments, the docking module interlace is configured to obtain and display the pump infusion history, including total medication delivered over time, timing of patient bolus requests and delivered patient boluses. In some embodiments, the pump and docking module, when integrated, communicate to each other through a wired connection upon pump mounting into the controller/container. In some embodiments, the pump and docking module, when integrated, communicate to each other through a wireless connection upon pump mounting into the controller/container. In some embodiments, the docking module includes a communication means configured to communicate from the system to a networked electronic health system. In some embodiments, the docking module is configured to hold and secure medication presented in a bag, semi-rigid container or syringe. In some embodiments, the docking module includes electronics and software to interface to external sensors comprising at least one of SpO2, EtCO2, minute ventilation and patient vital signs sensors, and wherein external sensor output is available through an externally-accessible interface of the docking module. In some embodiments, external sensor output is digested by an algorithm operable in the system to notify the pump user and/or automatically stop the pump under defined conditions of respiratory depression. In some embodiments, the system further comprises a database and at least one signal associating within the system the pump, a particular user of the pump, that user's medication, and the medication order. In some embodiments, the system further comprises a bar code scanner or near field sensing system. In some embodiments, the system further comprises a processor, mode algorithm, and mode indicator, collectively configured to modify a mode indicator to inform a user of the present mode, the modes linked to at least route of delivery or family of medication. In some embodiments, the docking module is further configured to indicate a current delivery mode using color coding. In some embodiments, an outer surface of one or more of the portable infusion pump and the pump docking module is configured to indicate a current delivery mode using color coding. In some embodiments, the pump docking module is configured to securely house the portable infusion pump by electronically recognizing and logging at least one of the following actions for a door of the pump docking module: locking, opening, closing, and unlocking. In some embodiments, the pump docking module is configured to securely house the portable infusion pump by electronically recognizing and logging at least one of the following actions: locking, attaching, detaching, and unlocking of the pump docking module to and from a pole.

### BRIEF DESCRIPTION OF THE DRAWINGS

FIG. 1 shows an infusion pump system in schematic form.

FIG. 1A shows a front perspective view of an example simplified infusion pump.

FIG. 1B shows a front plan view of an example ambulatory pump.

FIG. 2A shows a perspective view of a cassette-based infusion pump.

FIG. 2B shows a plan view of the front of the infusion pump of FIG. 2A.

FIG. 2C shows a plan view of the back of the infusion pump of FIG. 2A.

FIG. 2D shows a top view of the infusion pump of FIG. 2A.

FIG. 2E shows a side view of the infusion pump of FIG. 2A.

FIG. 3 shows a schematic diagram of a pump system having interacting features.

FIG. 4 shows an example hybrid ambulatory and bedside pump system.

FIG. 5A shows an interior view of an open hybrid pump and lockbox system.

FIG. 5B shows an exterior view of a closed configuration of the hybrid pump and lockbox system of FIG. 5A.

FIG. 6 shows an advanced hybrid lockbox and pump system with transparent aspects.

FIG. 7A shows an advanced hybrid lockbox and pump system with a slide-out feature.

FIG. 7B shows another view of the system of FIG. 7A.

FIG. 8 shows a hinged configuration for a hybrid pump and lockbox.

FIG. 9 shows a nested configuration for a hybrid pump and lockbox.

### DETAILED DESCRIPTION

Patients all over the world who are in need of medical care would benefit from infusion therapy (e.g., parenteral and/or intravenous infusion), not only during surgery or when hospitalized, but also during recovery when they are more mobile. Described concepts are intended for intravenous use as well as in epidural and other regional applications, as well as subcutaneous infusion. Parenteral infusion can be used, as well as enteral infusion and continuous irrigation.

Infusion (especially intravenous infusion) generally involves inserting a needle into a patient's blood vessel, usually in the hand or arm, and then coupling the needle to a catheter in communication with one or more different types of therapeutic fluids. Once connected, the fluid travels from the fluid source(s), through the catheter, and into the patient. The fluid can provide certain desired benefits to the patient, such as maintaining hydration or nourishment, diminishing infection, reducing pain, lowing the risk of blood clots, maintaining blood pressure, providing chemotherapy, and/or delivering any other suitable drug or other therapeutic liquid to the patient. Electronic infusion pumps in communication with the fluid sources and the patient can help to increase the accuracy and consistency of fluid delivery to patients. Further, medication and fluid infusion can be directed through other IV access devices such as central venous catheters, peripherally inserted catheters or through non-IV routes, such as into subcutaneous or regional (such as epidural) space or via the enteral pathway. Thus, infusion pumps are important medical devices that provide and control a flow of fluid to a patient. These can be used for patients that are in bed, and those that are well enough to move more freely. However, such different mobility contexts present different needs and challenges.

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

This specification provides textual descriptions and illustrations of many devices, components, assemblies, and subassemblies. Any structure, material, function, method, or step that is described and/or illustrated in one example can be used by itself or with or instead of any structure, material, function, method, or step that is described and/or illustrated in another example or used in this field. The text and drawings merely provide examples and should not be interpreted as limiting or exclusive. No feature disclosed in this application is considered critical or indispensable. The relative sizes and proportions of the components illustrated in the drawings form part of the supporting disclosure of this specification, but should not be considered to limit any claim unless recited in such claim.

The present disclosure provides for a multi-function infusion pump accessory and methods of use in conjunction with an associated infusion pump. The present disclosure also provides for a hybrid pain management and ambulatory pump platform and relates to infusion devices and accessories. For example, a next generation ambulatory infusion platform and associated accessories can meet the divergent needs of a small mobile ambulatory pump and the in-facility desire for increased screen size and enhanced user interface and product functionality in patient-controlled analgesia (PCA), patient controlled epidural analgesia (PCEA), programmed intermittent epidural bolus (PIEB) and/or large volume (LV) embodiments. A “multi-function” accessory can refer to various features and capabilities discussed herein. For example such an accessory can comprise an advanced lockbox for a modular pump that interacts with the pump (to control or display information therefrom) and presents a relatively large user interface on the surface of the accessory.

For example, in some embodiments, a combination of an ambulatory infusion pump and multi-function accessory can enable a small pump to be used in more traditional pole mounted role such as with PCA or general infusion. Thus, an integrated accessory can combine functions that are currently not available for ambulatory pumps. Functionality of the following devices can be consolidated: a supplemental screen/user interface, lock box, a docking station, a rechargeable battery, an AC power cord, a patient controlled bolus cord (e.g. an “extending patient control,” or a patient control that contains a simplified control interface such as a single button and extends away from a pump for easier access by a patient or user, for example), a pole clamp, a wired and wireless connection capability, advanced control and electronic memory—e.g., to interface to captive or third party sensing (respiratory monitoring, infiltration detection, patient vitals)—and physical storage capacity. Through this product design approach, the most appropriate features necessary for market leading ambulatory and pole-mounted PCA. PCEA and/or PIEB pumps are met in a single system (e.g., a pump and an advanced lockbox). Consolidation of some features and functions in an accessory (e.g., a stationary or bedside device) can of load some or all of these functions from a modular pump. Thus, a multi-function accessory can enable a simplified or streamlined pump, which can be readily or conveniently portable (e.g., ambulatory).

The present disclosure solves numerous problems. Ambulatory pumps are desired to be small when worn or carried in an ambulatory fashion by a patient, such as in a home or alternate site location or when used as a pain management device for patients in various settings. However, more traditional “pole mounted” LV and PCA pumps are typically larger devices, owing to technology constraints but also to the utility of larger user interfaces, enhanced battery capacity, and the desire to manage multiple infusion channels from an integrated device. The present disclosure describes one pump (“ambulatory” in traditional terms) that meets the broad infusion needs typically only addressed through a portfolio of pumps.

The present disclosure addresses the needs and challenges of different mobility contexts, in part, by describing how functionality can be divided between different structures and used intermittently, alternatively, or cumulatively. For example, battery and plug-in power can be integrated effectively with complementary structures. A smaller device can be enclosed in a larger device that electronically integrates with it while providing different feature combinations (e.g., power management, user interface, physical security, and/or cyber security). The smaller device can be more portable, while the larger device is more secure. The smaller device can be operated both independently of, and in connection with, the larger device.

Infusion pump hardware can be packaged and arranged in different ways for a variety of functions and purposes. For example, a “stationary pump” (or other stationary devices or modules) can be configured mainly for use at or near a patient's bedside. Stationary pumps can be very large and heavy because they can have large displays, long battery life (and therefore large batteries), highly durable pumping components intended for long and continuous use by many different patients, bulky protective housings, and many electronic components for storing software and data and for communicating wirelessly with hospital record systems. Stationary pumps can be attached to mounting poles that have wheels or can be movable in other limited ways. In other words, a stationary pump need not be strictly stationary at all times. Fluid source containers, such as intravenous (IV) infusion bags, can be attached to these poles as well, positioned above the stationary pumps. The contents of these bags can be in fluid communication with the stationary pumps by way of fluid lines. A stationary pump mounted on a pole with wheels can be pushed along by a user while he or she walks short distances, such as between the user's bed and the bathroom, or in the hallway of a hospital, or between rooms in a home. However, a stationary pump may be too large, heavy, and/or bulky to be conveniently carried directly on the patient's body or clothes during infusion, or to permit a patient to conveniently move long distances with it during infusion (e.g., while going for a walk outside, riding in a car, or going shopping). An example of a stationary pump is the Plum 360 infusion pump sold by ICU Medical. Inc., of San Clemente, California.

Pumps that can be worn or carried on the body or clothes of the user and that can be conveniently moved long distances with the user can be referred to as “portable pumps” or “ambulatory pumps,” in some embodiments, portable or ambulatory pumps can be small enough to be held or carried in one hand or to fit in a pocket or be carried clipped to a belt or held in a sling or pouch or attached by a lanyard or tether on a person's body. An example portable or ambulatory pump is the CADD-Solis infusion pump, available from ICU Medical, Inc. of San Clemente, California.

Infusion pump hardware can also be packaged and arranged such that an analgesic substance is accessible in increments, so that the patient can personally control and manage pain. (However, such medication can also be limited in how much is dispensed over a certain time period to a patient, notwithstanding some patient control). Such systems can be referred to as patient-controlled analgesia (“PCA”) pumps. Morphine and hydromorphone can be used for such applications and are typically secured. Further, opioid “cocktails”, (e.g., ropivacaine and fentanyl) can be used for used for epidural applications and may or may not be secured. Subcutaneous high dose opioid administration is also sometimes used in hospice, both acute-care and alternate-site. Thus, parenteral PCA pain management can advantageously use a lockbox or other security measures to protect the controlled substances (e.g., those containing opioids) from diversion and is particularly useful in acute care settings. Labor and delivery is an advantageous location for a secured approach for epidural delivery because the patient frequently has a spouse in the room. The security often required for PCA pain management drugs is not always compatible with ambulatory use, which seeks to minimize pump size. For example, a pump delivering an epidural may be in a lockbox, or carried in a bag/fanny sack which could be a less secure system, though more amenable to a mobile patient. An ambulatory pump can further be used by a patient receiving a home care infusion.

However, as disclosed herein, an advanced lockbox can be integrated with an ambulatory infusion pump, supporting the competing needs of a small, simple ambulatory pump for alternate site care with the more advanced features helpful in an acute care personally controlled analgesic (“PCA”) parenteral delivery system. An advanced or smart lockbox can be used with a removable, portable pump. Offloading features to the smart lockbox can help minimize the pump size and minimize pump interface requirements to meet ambulatory needs, and can support all the complexity and security which may be required of a PCA pump. Locating functionality in a lockbox can allow the pump itself to be minimized in size and complexity. For example, a smart lock box can include a larger touchscreen (or other more sophisticated interface); more connectivity (e.g., wi-fi); AC power; more battery power; interfaces and support for patient sensors; and additional functionality.

### Syringe Pump Systems

In some embodiments, a pump system can include a reusable pump driver and a disposable fluid holder, such as a fluid cassette, syringe, section of tubing, etc. In a syringe pump example, a disposable syringe, which is typically adapted to be used for a single patient and/or for a limited time, is usually formed of plastic and has at least one inlet and an outlet respectively connected through flexible tubing to the fluid supply container and to the patient receiving the fluid. An actuator mechanically connected to a motor can urge a plunger in the syringe to move forward in a slow, controlled manner, thereby dispensing the medial fluid. A syringe arrangement is often useful for patient-controlled analgesic (PCA) pumps. Syringes can be used to precisely measure and manage analgesic volumes, through visual inspection of graduated markings on a syringe cylindrical volume, for example. Syringes can be inserted into a clear container that can provide visual inspection and security benefits. A syringe pump example is the LifeCare PCA™ Infusion System, available from ICU Medical.

The described self-sufficient or ambulatory pumps referred to herein may use any type of pumping mechanism. For example, they can use a single use cassette and/or a peristaltic pumping mechanism on tubing (e.g., silicone or PVC tubing) to deliver medication. The following provides one example but the disclosed inventions are not limited to this example.

### Cassette Pump Systems

Infusions pumps also can use a cassette. In a cassette pump example, a disposable cassette, which is typically adapted to be used for a single patient and/or for a limited time, is usually a small plastic unit having at least one inlet and an outlet respectively connected through flexible tubing to the fluid supply container and to the patient receiving the fluid. In some embodiments, the cassette can include a pumping chamber. The flow of fluid through the chamber can be controlled by a plunger or pumping element activated in a controlled manner by the reusable pump driver. For example, the cassette chamber can have one wall formed by a flexible diaphragm against which the plunger is repeatedly pressed in a reciprocating manner, which causes the fluid to flow. The pump driver can include the plunger or pumping element for controlling the flow of fluid into and out of the pumping chamber in the cassette, and it may also include one or more controls and/or vents to help deliver the fluid to the patient at a pre-set rate, in a predetermined manner, for a particular pre-selected time, and/or at a pre-selected total dosage.

In cassette embodiments, the fluid can enter a cassette through an inlet and can be forced through an outlet under pressure. The fluid is delivered to the outlet when the pump plunger forces the membrane into the pumping chamber to displace the fluid. During the intake stroke, the pump plunger draws back, the membrane covering the pumping chamber retracts or pulls back from its prior inwardly displaced position, and the fluid is then drawn through the open inlet and into the pumping chamber. In a pumping stroke, the pump plunger forces the membrane back into the pumping chamber to force the fluid contained therein through the outlet. Some pumps can thus use passive valves to support pumping. However, active valves can also be timed to open or close simultaneously with appropriate portions of a pumping stroke cycle. By repeating the pumping actions in an electronically controlled manner, the fluid flows into and out of the cassette. Typically at lower rates, during the pumping cycle, the pump plunger is stepped in a specified timing sequence to deliver successive pulses of fluid from the pump chamber. For example, a single draw motion can correspond to a long series of small expel motions. Thus, the fluid can now from the cassette in a series of spaced-apart pulses rather than an uninterrupted flow. When the pulses occur in rapid succession, the flow approximates a continuous flow. At higher rates the pump can typically displace fluid in a smoothly continuous manner. A cassette pump example is the Plum 360™ Smart Infusion System, available from ICU Medical.

### Example Simplified Infusion Pump

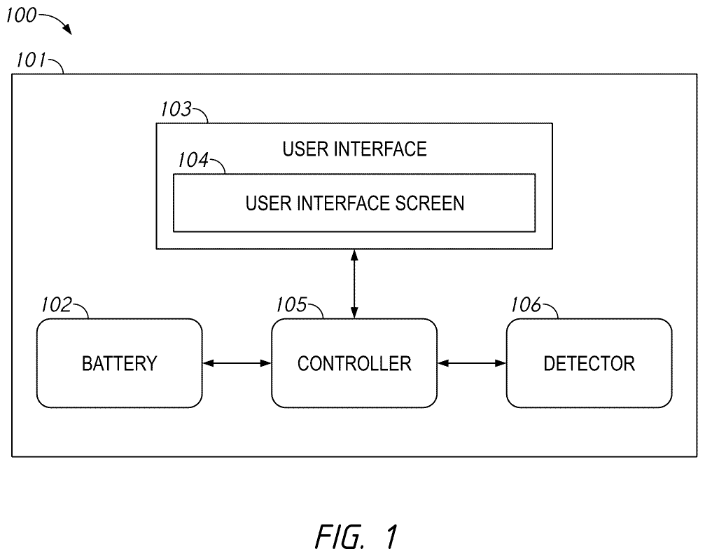

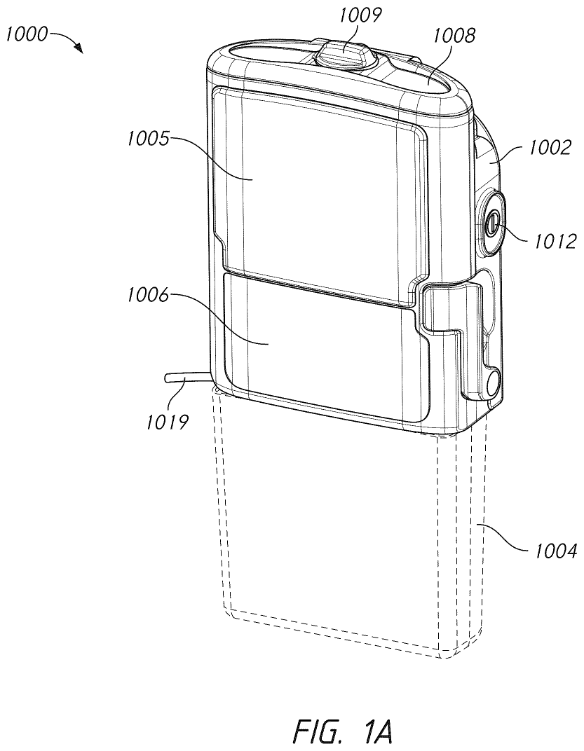

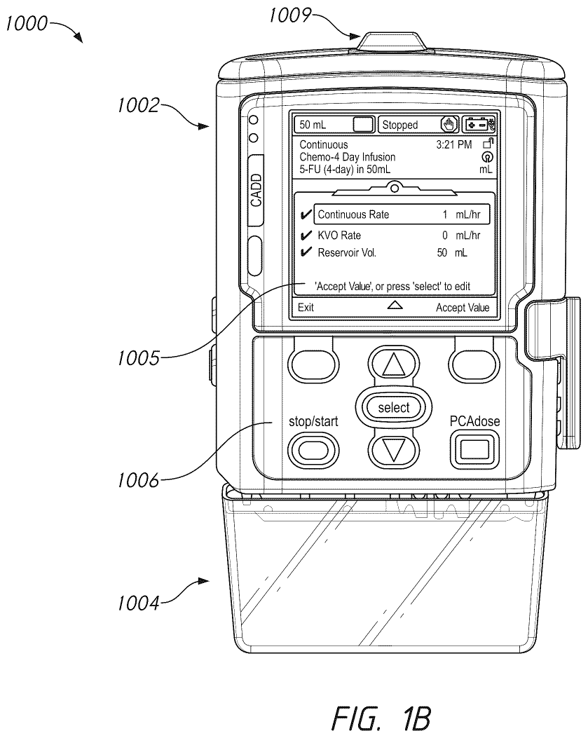

FIG. 1 shows a diagram of an infusion pump system 100 including an infusion pump 101 that infuses fluids that may include drugs, nutrients, blood or other liquids into a patient. In at least one embodiment, the infusion pump 101 includes one or more rechargeable batteries 102, a user interface 103 and a user interface screen 104, an infusion pump controller 105, and, optionally, a detector 106 (e.g., a device configured to detect ambulation of the patient). In one or more embodiments, the infusion pump controller 105 includes a computer and is directly or indirectly coupled with the user interface 103 and the user interface screen 104.

FIG. 1A illustrates a perspective view of an infusion pump 1000. A control module 1002 and an optional reservoir cassette 1004 can be included. Infusion pump 1000 can be a CADD® (Computerized Ambulatory Drug Delivery) Ambulatory Infusion Pump system from ICU Medical. Control module 1002 can include a user interface having a display screen 1005 and a control pad 1006 (buttons, etc., of the control pad are not illustrated). In some embodiments, an enlarged screen can be provided to enhance display of bolus history information, etc.

Control module 1002 can also include a battery door 1008, including a knob 1009 for locking and unlocking the door 1008, which can cover a battery compartment in which batteries for powering the pump 1000 can be housed. In some examples, a combination battery and wireless communication module can be present approximately where battery door 1008 is illustrated. Control module 1002 can also include a power switch 1012.

Example infusion pump 1000 can include a replaceable reservoir cassette 1004 connected to control module 1002. A reservoir cassette 1004 can house a reservoir that in turn can contain an infusate to be delivered to a patient. (Note that the reservoir cassette 1004 is illustrated with dashed lines in FIG. 1A to demonstrate that some embodiments do not have this cassette. In such cases, a pump can connect with a more traditional infusion set that mounts to the bottom of the pump, in place of cassette 1004, which in turn can draw from a bag that can be locked into a lockbox). Tubing 1019 can extend from the cassette 1004 and fluidly communicate with an infusion set or catheter (not shown) to deliver the infusate to the patient. Tubing 1019 can be fluidically coupled to such an infusion set or catheter with a Luer-type connector or any other suitable connector (not shown). The control module 1002 can be used to control the flow of infusate from cassette 1004. One example of such a cassette is the CADD® Medication Cassette Reservoir (available in various reservoir volumes) from ICU Medical. Such a pump can use a peristaltic approach, with a mechanical interface to urge fluid flow through an accommodating tube. An example of peristaltic pump mechanics can be seen in U.S. Pat. No. 4,650,469 to Berg et al., the entirety of which is hereby incorporated by reference, for all purposes.

FIG. 1B illustrates a plan view of an example CADD ambulatory pump. Like the example of FIG. 1A, this embodiment can include a control module 1002 with a user interface having a display screen 1005 and a control pad 1006 with buttons. A knob 1009 can be used for locking unlocking a door to a battery compartment in which batteries for powering the pump 1000 can be housed. A replaceable reservoir cassette 1004 can house a reservoir that in turn can contain an infusate to be delivered to a patient.

Ambulatory pumps such as those illustrated herein can internally include and hold one or more rechargeable batteries and an infusion pump controller. In one or more embodiments, the infusion pump apparatus may include a speaker, a user interface that may include a screen, for example a touch screen, one or more buttons and, may internally include and hold a computer, microprocessor, controller or any other type of programmable apparatus that may be coupled with the user interface.

In some embodiments, an infusion pump such as those depicted in FIGS. 1, 1A, and 1B can function as an ambulatory pump. For portable pumps, battery capacity management and charging can be important. Accordingly, the entire disclosure of U.S. Pat. No. 9,333,291, titled infusion Pump Battery Capacity Management and Battery Charge Alert System and Method is incorporated by reference, for all purposes.

With further reference to FIG. 1, in some embodiments, at least one detector 106 is configured to detect ambulation of the patient. The detector may include one or more of an input power sensor, an accelerometer and a wireless network interface. By way of one or more embodiments, the at least one detector 106 that detects the ambulation of the patient may include a sensor that detects removal of a power input from the infusion pump. In one or more embodiments, the accelerometer may be coupled with the computer such that the accelerometer detects acceleration indicative of ambulation of the patient coupled with the infusion pump. In at least one embodiment, the wireless network interface is coupled with the computer such that wireless interface detects wireless connectivity changes of the infusion pump 101 coupled with the patient.

According to one or more embodiments, during a programmed infusion, the at least one detector 106 may detect ambulation based on removal of a power input from the infusion pump 101 for less than 5 minutes, and one or more of sustained infusion pump movements for at least 10 seconds detected by the accelerometer, and infusion pump movement based on 3 events of wireless connectivity change detected by the wireless network interface. Other thresholds for these events in terms of the time duration or acceleration values or wireless connection changes are in keeping with the invention. For example, if the input power is lost as detected by the input power sensor, and the accelerometer detects accelerations over a predetermined threshold, or for example when integrated or double integrated show a velocity or movement over respective predetermined thresholds, or if wireless interface detects a number of wireless network changes over a predetermined threshold, then the patient ambulation may be indicated. In one or more embodiments, if any combinations of these values is detected or correlated, then ambulation may be indicated, and battery calculations may be undertaken in order to enhance battery performance by power conservation and or provide an alert related to battery capacity. For example, when the ambulatory pump has determined that patient ambulation is occurring, the ambulatory pump can convert to a power saving mode by, for example, reducing the frequency of wireless communication, reducing the illumination level of the display's backlight, etc.

### Example Cassette-Based Infusion Pump

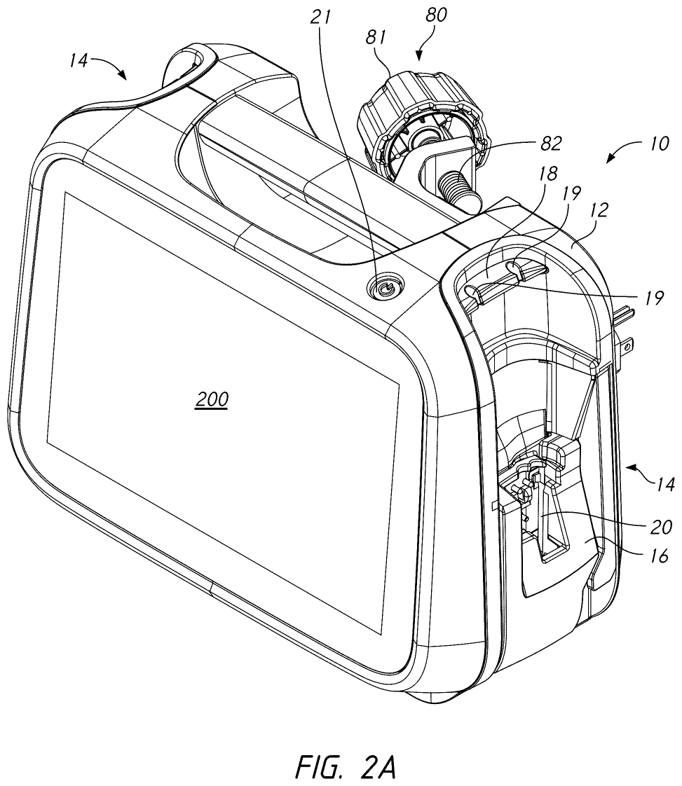

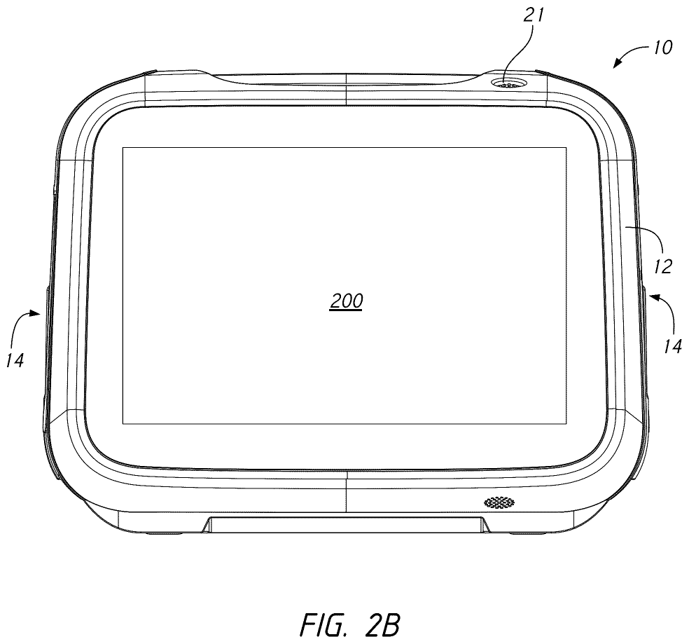

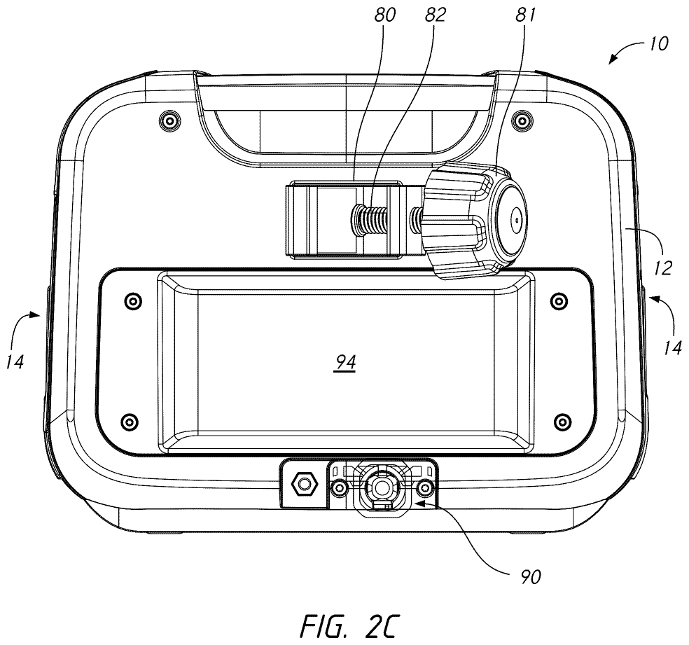

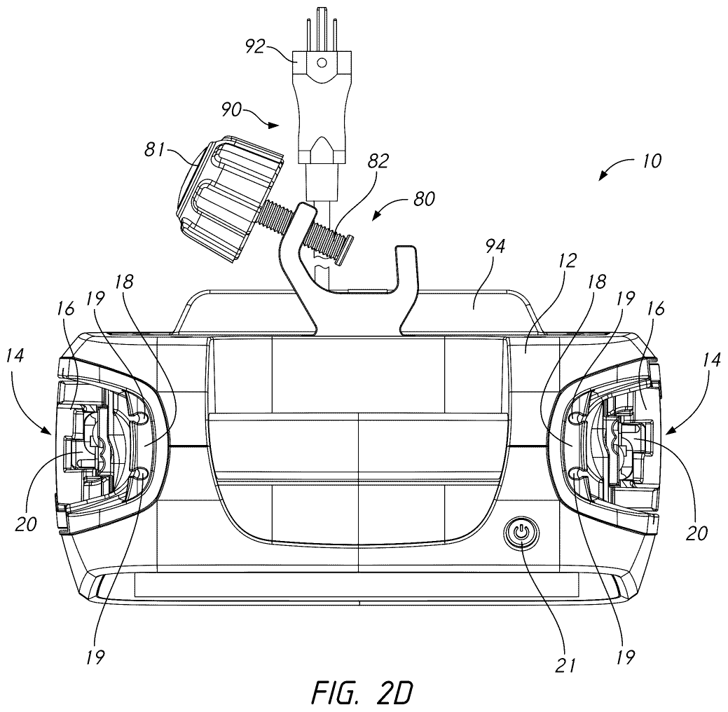

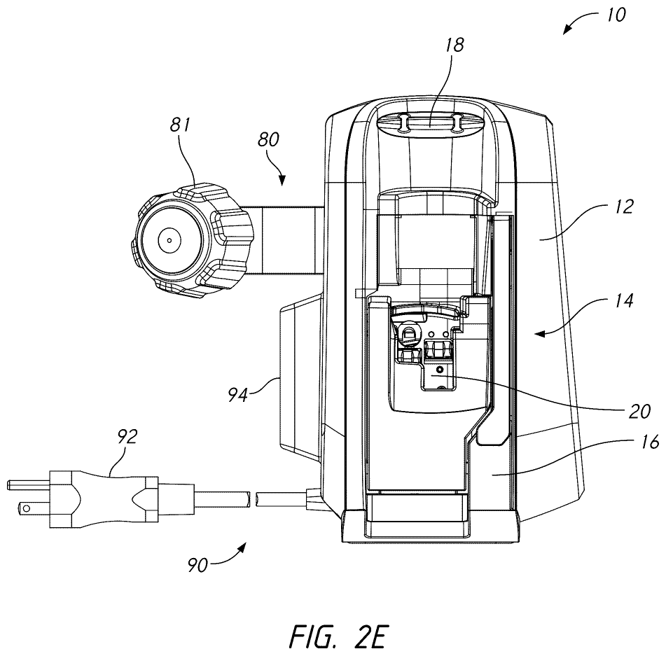

FIGS. 2A-2E show an electronic medical intravenous pump 10 with a housing 12 and at least one pump driver 14 attached to the housing 12. As illustrated, a plurality of pump drivers 14 (e.g., at least two) can be integrally provided within the same housing 12 of a single medical pump 10. Either or both of the pump drivers 14 can include a cover 16 that partially or entirely encloses an outer surface of the pump driver 14, an indicator 18 (e.g., an illuminating communicator) attached to the cover 16, one or more tube holders 19, and a loader 21) configured to securely receive and releasable hold a disposable fluid holder, including but not limited to a cassette, syringe, and/or tubing. The one or more tube holders 19 can be configured to removably receive and securely hold one or more fluid-conveying tubes extending into or exiting from fluid holder when the fluid holder is received into the loader 20. The indicator IN can communicate one or more messages to a user, such as by temporarily illuminating in one or more colors. Examples of one or more message include confirming that a pump driver 14 near the indicator is currently active and pumping or that one or more instructions being received from a user will apply to a pump driver 14 near the indicator 18. The loader 20 can be a mechanism with multiple moving parts that opens, closes, expands, contracts, clasps, grasps, releases, and/or couples with the fluid holder to securely hold the fluid holder on or within the pump 10 during fluid pumping into the patient. The loader 20 can be integrated into and positioned on or within the pump 10 near the cover 16 adjacent to the indicator 18.

A user communicator, such as display/input device 200, can be provided to convey information to and/or receive information from a user (e.g., in an interactive manner). As illustrated, the user communicator is a touch screen that is configured to provide information to a user through an illuminated dynamic display and is configured to sense a user's touch to make selections and/or to allow the user to input instructions or data. For example, the display/input device 200 can permit the user to input and see confirmation of the infusion rate, the volume of fluid to be infused (VTBI), the type of drug being infused, the name of the patient, and/or any other useful information. The display/input device 200 can be configured to display one or more pumping parameters on a continuing basis, such as the name of the drug being infused, the infusion rate, the volume that has been infused and/or the volume remaining to be infused, and/or the elapsed time of infusion and/or the time remaining for the programmed course of infusion, etc. As shown, the touch screen can be very large, for example at least about 4 inches×at least about 6 inches, or at least about 6 inches×at least about 8 inches. In the illustrated example, the touch screen fills substantially the entire front surface of the pump 10 (see FIG. 1A), with only a small protective boundary surrounding the touch screen on the front surface. As shown, the touch screen comprises at least about 80% or at least about 90% of the surface area of the front of the pump 10. In some implementations, the front of the touch screen comprises a clear glass or plastic plate that can be attached to the housing 20 in a manner that resists liquid ingress, such as using a water-proof gasket and/or adhesive that can withstand repeated exposure to cleaning and sanitizing agents commonly used in hospitals without significant degradation.

An actuator 21 can be provided separate from the user communicator. The actuator 21 can be configured to receive an input and/or display information to a user. As shown, the actuator 21 is a power button that permits the user to press on the actuator 21 to power up the pump 10. The actuator 21 can illuminated to communicate to the user that the pump 11) is power on. If the power source is running low, the actuator 21 can change the color of illumination to quickly show to a user that a power source needs to be replenished.

In some embodiments, the user communicator, such as a display/input device 200, can alternatively or additionally comprise one or more screens, speakers, lights, haptic vibrators, electronic numerical and/or alphabetic read-outs, keyboards, physical or virtual buttons, capacitive touch sensors, microphones, and/or cameras, etc.

During use, the pump 10 is typically positioned near the patient who is receiving fluid infusion from the pump 10, usually lying in a bed or sitting in a chair. In some embodiments, the pump 11 may be configured to be an ambulatory pump, which will typically include a smaller housing, user communicator, battery, etc., so as to be conveniently transportable on or near a mobile patient. In many implementations, the pump 10 is attached to an IV pole stand (not shown) adjacent to the patient's bed or chair. As shown, the pump 10 can include a connector KU that is configured to removably attach the pump 11 to the IV pole stand. As shown, the connector NO can comprise an adjustable clamp with a large, easily graspable user actuator, such as a rotatable knob 81 that can be configured to selectively advance or retract a threaded shat 82. At an end of the shaft 82 opposite from the knob 11 is a pole-contacting surface that can be rotatably advanced by the user to exert a force against a selected region of the pole, tightly pushing the pole against a rear surface of the pump 10, thereby securely holding the pump 10 in place on the pole during use. The selected region of the pole where the contacting surface of the shaft 212 is coupled can be chosen so as to position the pump 10 at a desired height for convenient and effective pumping and interaction with the patient and user.

The pump 10 can include a power source 90. In some embodiments, the power source can comprise one or more channels for selectively supplying power to the pump 10. For example, as illustrated, the power source 90 can comprise an electrical cable 92 configured to be attached to an electrical outlet and/or a portable, rechargeable battery 94. One or more components of the pump 10 can operate using either or both sources of electrical power. The electrical cable 92 can be configured to supply electrical power to the pump 10 and/or supply electrical power to the battery 94 to recharge or to maintain electrical power in the battery 94.

Inside of the housing 20 of the pump 10, various electrical systems can be provided to control and regulate the pumping of medical fluid by the pump 10 into the patient and/or to communicate with the user and/or one or more other entities. For example, the pump 10 can include a circuit board that includes a user interface controller (UIC) configured to control and interact with a user interface, such as a graphical user interface, that can be displayed on the user communicator or display/input device 201. The pump 10 can include a printed circuit board that includes a pump motor controller (PMC) that controls one or more pump drivers 14. In some embodiments, the PMC is located on a separate circuit board from the UIC and/or the PMC is independent from and separately operable from the UIC, each of the PMC and UIC including different electronic processors capable of concurrent and independent operation. In some embodiments, there are at least two PMC's provided, a separate and independent one for each pump driver 14, capable of concurrent and independent operation from each other. The pump 10 can include a printed circuit board that includes a communications engine (CE) that controls electronic communications between the pump 10 and other entities (aside from the user), such as electronic, wired or wireless, communication with a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc. The CE can include or can be in electronic communication with an electronic transmitter, receiver, and/or transceiver capable of transmitting and/or receiving electronic information by wire or wirelessly (e.g., by Wi-Fi, Bluetooth, cellular signal, etc.). In some embodiments, the CE is located on a separate circuit board from either or both of the UIC and/or the PMC(s), and/or the CE is independent from and separately operable from either or both of the UIC and/or the PMC(s), each of the PMC(s), UIC, and CE including different electronic processors capable of concurrent and independent operation. In some embodiments, any, some, or all of the UIC, CE, and PMC(s) are capable of operational isolation from any, some, or all of the others such that it or they can turn off, stop working, encounter an error or enter a failure mode, and/or reset, without operationally affecting and/or without detrimentally affecting the operation of any, some, or all of the others. In such an operationally isolated configuration, any, some, or all of the UIC, CE, and PMC(s) can still be in periodic or continuous data transfer or communication with any, some, or all of the others. The UIC, PMC(s), and/or CE can be configured within the housing 20 of the pump 10 to be in electronic communication with each other, transmitting data and/or instructions between or among each of them as needed.

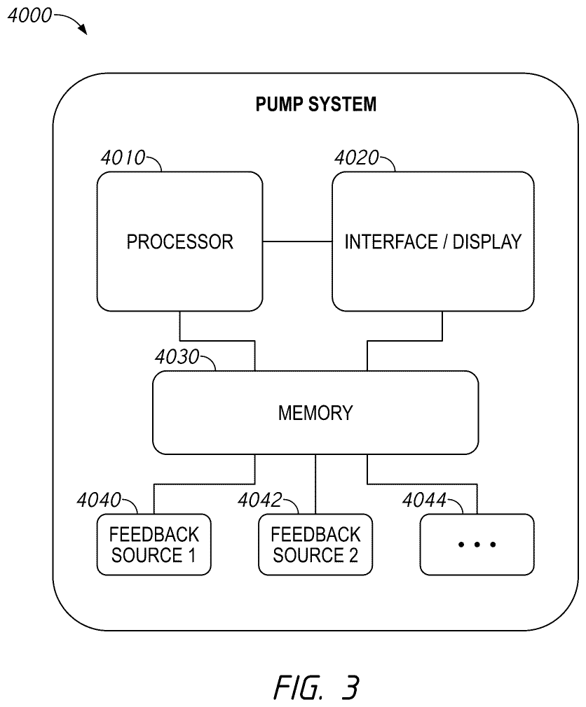

FIG. 3 shows a system 4000. The system 4000 can be a self-aware infusion pump and display system. Such a system can include a processor 4010 and a memory 4020. These can be configured to establish a known infusion volume history. Such a history can be important to help secure controlled substances and comply with legal and medical constraints, for example. Further, the log history of requested and delivered patient activated boluses and total medication amount delivered by hour provides insight into the pain level of the patient over time. A bolus history can be stored and compared to a medication prescription to confirm that rate and dosage limits have been followed. A system such as those described can be configured to interact with electronic medical records or other information systems to perform such checks or audits automatically, or upon demand. Reporting information can be provided using an enlarged screen or interface on a multi-function accessory (e.g., a pump docking enclosure such as those describe herein), and may include graphs, charts or other presentation means. The history can also be established by using system information regarding the duration of any pauses (e.g., those due to alarms, air bubble clearance, kink removal, or infusion bag, reservoir, etc.). The system can have a processor configured to use the known infusion volume history and an electronically stored drug database (e.g., that includes characteristic half-lives for particular drugs) to calculate present effective expected drug concentration. This can be conveyed to a clinician as a percentage of a desired (e.g., steady state) level—for example, showing a clinician that a patient's calculated drug level is 80% of the target equilibrium steady state. The system can also have a display configured to display to a clinician or other user the present effective calculated drug concentration, for example. The system may also distinguish; presentation of a delay in the medication reaching the patient (where no patient response should yet be expected); the medication reaching the patient but not yet expected to result in an equilibrium or steady state level in the patient; an expected steady state; and/or a temporary deviation from steady state that is being re-established. The system may also individually track the infusion volume history for each individual drug when contained in a combination medication (e.g., Ropivacaine 0.125% with fentanyl 2 micrograms/mL) that is delivered in mL/hour.

The system 4000 can provides an example structure as schematically illustrated. A processor 4010 can interact with an interface/display 4020. Both of these components can interact with a memory 4030, which can include a drug database and can store a pump/flow history. The memory can receive input from feedback source 4040, feedback source 4042, and additional feedback sources 4044. These feedback sources can include onboard sensors within the pump system itself, and they can include inputs from an interface/display 4020, provided for example by a user. These inputs can also include information regarding a patient or a medication, for example from a hospital information system that is connected via network to the pump system 4000.

The system 4000 can be further configured to calculate and display an estimated time when a drug will first reach a patient, an estimated time when a drug load or concentration will reach a specified target level for example within a patient, and/or an estimated time when the patient is expected to achieve a particular physiological response to the drug. The system can be configured to be self-aware in the sense that it knows its own history, its own constraints, and how these are most likely to affect the results within an infusate destination—for example a patient's bloodstream. The system can access electronic hospital records or other databases, for example, to correlate pain management medications administered to the patient through other delivery routes (e.g., pill, standard injection, inhaled and administered by an anesthesiologist during a precursor surgical procedure). A larger and faster processor, more efficient data transfer, space for communication modules, etc. (all enabled or otherwise enhanced by a larger security enclosure), can enhance a device's ability to address, perform, or convey such a correlation.

The system 4000 can be configured to compensate for pauses in delivery of an infusate. This can be accomplished by infusing larger amounts (e.g., catch-up continuous infusions or boluses) of a drug into a patient within safe boundaries for concentration and timing, or it can be accomplished by infusing a drug at a constrained rate for a set amount of time or until a particular infusion goal is achieved. The system processor 4010 and memory 4020 can be further configured to facilitate prediction of future drug concentration by calculating extrapolated data points based on a trend line or other inputs, and the display 4020 can be configured to provide a graph to communicate the data points or trend line to a user. The system can be further configured to facilitate prediction of future drug concentration and automatically suggest or implement a flow rate change to avoid an undesired predicted future drug concentration. Memory 4030 can include a patient profile or other information relating to a specific treatment protocol or clinical history for a particular recipient for the infusate.

A system can comprise a noninvasive drug concentration estimator pump. The pump can have a memory configured to store a drug library, which can include multiple (e.g., two, three or more) fields selected from the following group: drug name, concentration or container volume, dosing unit, lower limit, upper limit, catch-up rate or dose permission, maximum catch-up rate or dose, drug hair-life, drug expiration, and drug source. The memory can further be configured to store a patient profile having demographic, medical, or identifying data specific to the patient. The memory and/or one or more sensors or processors can be configured to track and record pump behavior. A processor can be configured to use the drug library, patient profile, and pump behavior to calculate predicted drug levels in the patient without input from in-vivo sensors. An interface can be configured to display the predicted drug levels and periodic pump behavior indicators. The pump behavior can be real-time input of forward fluid flow and paused fluid flow. Pump behavior can also be a measure or indicator of total volume infused in a variety of time and date formats (e.g., shift totals).

An infusion pump can be configured to accept feedback on and account for numerous categories of information relating to its function and the expected results from any substances it infuses. For example, a pump can provide information (e.g., based on its own history) of expected in-patient amounts. It can track and account for infusion tube details, saline or other fluid carrier or “keep vessel open” how effects, and any initial setup delays after infusion is initially requested. It can account for drug half-life (or more sophisticated pharmacokinetic and pharmacodynamic medication models), elimination factors, and physiological responses. It can account for infusion pauses, including for bag or fluid reservoir replacement, air-in-line or occlusion alarms, etc. It can display related time-based information (past history, future projections, current levels, expected arrival time, expected response time).

### Example Hybrid Pumps

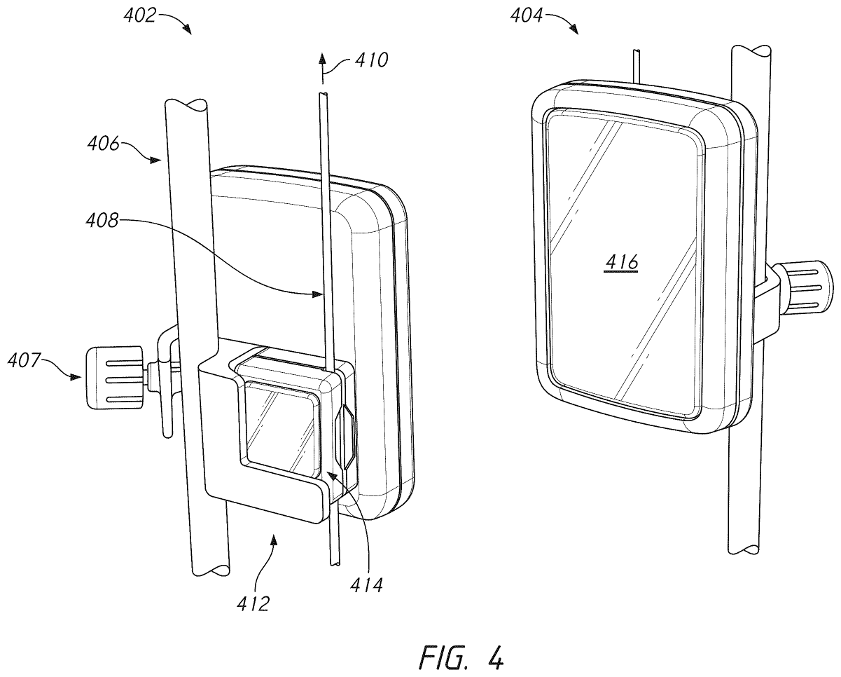

FIG. 4 shows an example pump 414 incorporated into a pump holder 412 at the back of a lock box system 402. An infusion line 408 runs through the pump 414 and the entire apparatus is secured to a pole 406 using a locking clamp and dial 407. In a front view 404, a large touchscreen display 416 is shown at the front of the lockbox system 402. In this configuration, a pump 414 can comprise an ambulatory or otherwise portable or small pump, such as a PCA pump. The infusion line 408 can connect to an infusate (e.g., IV) bag 410 that can also hang from the pole 406. Locking of bag and pump can be accomplished in numerous ways. In some embodiments, a pump administration set and/or fluid bag or reservoir can be protected from diversion beyond what is shown in FIG. 4.

The pump holder 412 can help align the pump for 14 with an electronic interface located at the back of the lock box system 402. Thus, a pump 414 can interface electronically with hardware contained within the lockbox system 402. Given such an interface, a display screen of a pump 414 can be synchronized and/or overridden by the display screen 416. The larger lock box system 402 can obtain information from the pump 414 that was stored while the pump 414 was being used in an ambulatory or portable mode. The lockbox system 402, and it's display 416, can assume control of the small portable pump 414 when it is plugged in and located in the holder 412. In this configuration, the pump 414 remains in contact with the infusion line 408 whether or not it is in captured/integrated or ambulatory mode. The present disclosure thus provides for dual mode and hybrid pump and container systems. The two modes can include a bedside mode and an ambulatory mode. The two modes can include a secured mode and a non-secured mode. The two modes can comprise portable mode and secured mode. The two modes can include a patient-control mode and a hospital control mode. The system can be a hybrid in the sense of fulfilling functionality of ambulatory and bedside pumps, with a single system. It can combine a stationary (e.g., primarily bedside) portion and a portable (e.g., ambulatory) portion, which can come together to function, and be readily separably for independent operation, in certain circumstances.

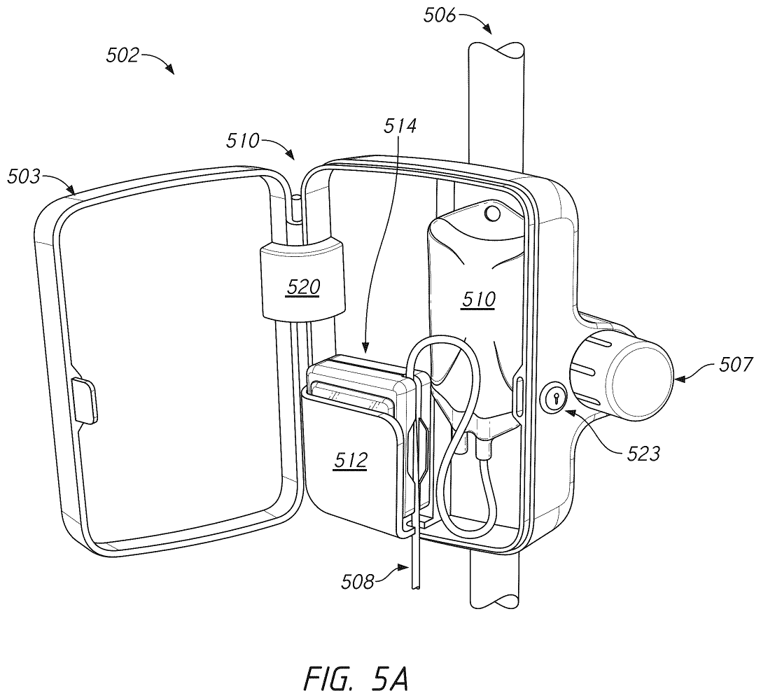

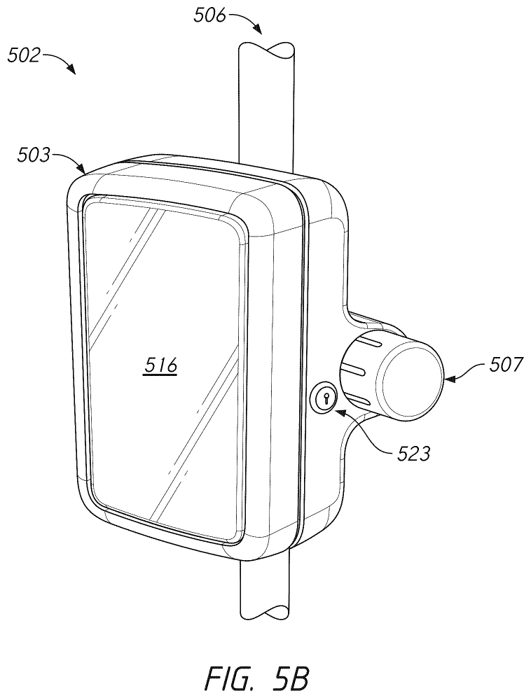

FIG. 5A shows how a lockbox system 502 can have a door 503 and a hinge arrangement 510 that can open to reveal the contents of the lockbox system 502. Within, a small portable pump 514 can be contained within a holder 512. An infusion line 508 can run through the pump 514 and connect with an infusate (e.g., IV) bag 510. The disclosed systems can deliver via various routes, such as epidural, and are not confined to standard intravenous (or IV) routes. The entire lock box system 502 can be secured to an IV pole 506 using a locking pole clamp 507. A mechanical or electronic door lock 523 can help secure the door 503 closed. A cord holder 520 can provide a passage through which electronic wiring can communicate between electronic circuit boards or other processor materials contained within the back of the lock box 502 and a screen or other processor materials contained within a door 503. This configuration has many benefits, including the security of containing both the ambulatory pump 514 and the medication or infusate bag 511) within a secure locked environment. This is also a simple construction that may be simple to explain to many healthcare providers.

FIG. 5B shows the structure of FIG. 5A, only with this time with the door 503 closed to reveal a large touchscreen display 516 integrated into the door 503. The door lock 523 is still visible securing the door in place. The dial 507 associated with a locking pole clamp is also visible. As shown in this figure, the pump and infusate bag are not within sight of a typical user, and therefore positioned relatively securely for use. The lock is shown here as a traditional mechanical (physical key) lock, but a lock can alternatively be activated by keypad entry of a passcode, swiping or a clinician badge, etc.

### Advanced Lockbox Features

Disclosed embodiments include an advanced lockbox that can help secure medication. This is especially helpful for analgesics, which can comprise controlled substances. A lockbox can deter diversion, for example. Advanced lockboxes can offload functions from a simplified pump. Many duplications of functions between a pump and a lockbox are contemplated. However, in preferred embodiments, a simplified pump can function independently from an advanced lockbox or be otherwise self-contained.

An advanced lockbox can comprise an “accessory” to a pump (as described below). An advanced lockbox can comprise a pump docking module. An advanced lockbox can form a stationary docking station. Stationary may refer to a larger device that is mounted on a wheeled pole or other holder but is nevertheless less portable than another related device. The advanced lockbox can hold or support or secure a smaller or modular and portable pump device. The pump device can be removed or disengaged from the lockbox in a non-technical manner, such as without removing a housing or panel or other component thereof, without tools, and/or by a person without technical training. For example, in some embodiments and/or modes, rapid, non-technical disengagement can comprise a hospital user rapidly and conveniently detaching the pump manually (e.g., by unsnapping, gently lifting, unlatching, using a simple button or other actuator, opening a lid, etc.). Rapid disengagement can occur in normal use in less than or equal to about 20 seconds. Non-technical disengagement can include steps such as opening a door or opening a hinged enclosure to access and remove the pump device before disconnection of the pump device from the docking station. In some embodiments, rapid, non-technical disengagement can be accomplished in a single disconnection action or motion, without requiring separate steps for detaching the housing of the pump device and also separately detaching one or more wires, connectors, leads, fluid tubes, etc. in a different action or motion. Authorization for pump detachment or disengagement can be strictly enforced (e.g., through biometric or other locks, etc.), while still making the physical act of detachment relatively rapid and straightforward, once authorized (e.g., once a lock is released).

In some embodiments, an advanced lockbox can include one or more of the following features or structures: a large touch-screen graphic user interface (GUI); AC power; a bolus cord; Wi-Fi; light source to illuminate the lockbox a desired color a system. sensors, controls or alarms for monitoring locking and unlocking of the box; a system, sensors, controls or alarms for monitoring locking and unlocking of the box from a mount such as an IV pole.

Variations of an advanced lockbox can include (in addition to or as an alternative to the above), one or more of the following. The advanced lockbox front can expose all, some or none of the ambulatory pump itself. The advanced lockbox front can expose all, some or none of the medication container. The term “medication container” can include a container for a medication reservoir, for example. The advanced lockbox GUI can assume control of the pump, with pump GUI off or complementing the lockbox GUI. The communication between the pump and advanced lockbox can be wired, or wireless (such as Bluetooth). Wired (direct connection) interfaces such as AC power, bolus cord, respiratory monitor. USB or other communication ports can remain directly connected to the ambulatory pump even if it is contained within the advanced lockbox (and therefore have access holes or recesses allowing cords to protrude, for example). Alternatively or additionally, these features can be connected to and extend from the advanced lockbox.

An advanced lockbox can be ruggedized to deter diversion, avoid breakage, improve security, and/or meet hospital or other standards. Various types of containers (e.g., medication or other infusion fluid containers) can be supported by one or more configurations. Such containers include bags, semi-rigid containers, rigid reservoirs, syringes or vials. Pumping structure and features can provide a parameter for determining the containers that can be used with a particular lock box. Geometry of an advanced lock box can also help determine which containers can be used. An advanced lockbox can specifically for IV PCA include space (inside or outside of the locking area) for a second pump to deliver diluent or carrier fluid, with pumps loaded in the “pain” or “carrier” locations automatically assuming those roles to enhance or improve safety.

Some pumps can be specifically designed for (or, if restriction is desired, only allow or support) particular therapies-such as IV PCA. Subcutaneous PCA, Peripheral Nerve Block or Neuraxial (e.g., epidural and intrathecal modes)—when loaded into a lockbox. This constraint can be physical or software-based. A particular mode or restriction status can be evident to a user. Visual or audible information can be provided regarding a particular mode of a pump or system containing a pump. For example, a touchscreen background color can reflect the delivery mode, and/or the lockbox could be illuminated by color to reflect delivery mode. Modes can also be indicated with text or other indicators on a touch screen or with a physical switch or flag. In addition to indicating a mode, a touchscreen or other visible element can provide other information. For example, a touchscreen can display patient parameters: respiratory data such as SpO2, EtCO2, minute ventilation, other respiratory sensor output, infiltration detection or other vital signs. Interfaces to captive or third party sensing (circuit boards) can be maintained in the lockbox, for example.

Disclosed embodiments (e.g., an advanced lockbox or related software) can provide for logging and presentation of event or medication history (such as timing of patient boluses requested and delivered via a patient bolus button and total medication amount delivered per hour). Pumps can show graphical representations of patient bolus requests and patient bolus deliveries, using an expanded screen to better appreciate and use this functionality. With a large lockbox including a color touch screen, such information can be shown in large easy to view graphical format.

### Additional Hybrid Pump Configurations

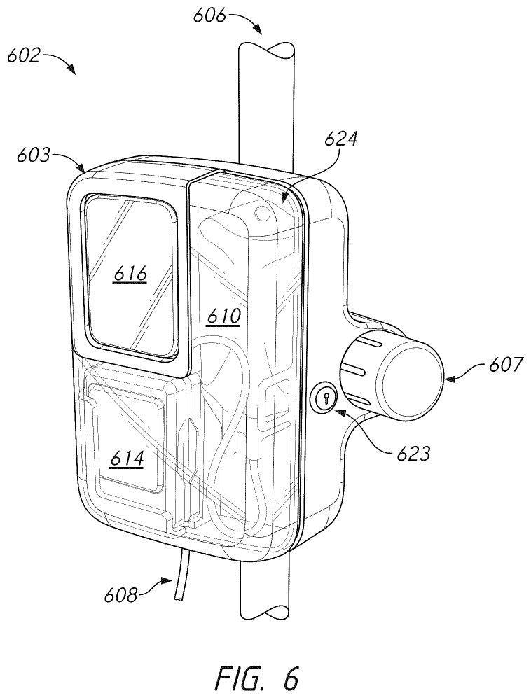

FIG. 6 shows another example of an integrated system 602 for combining security, infusion, and control benefits. An infusion bag may be visible, while a pump may or may not be visible. The system 602 can be positioned on an IV pole 606 using a locking pole clamp controlled by a dial 607. A touch screen display 616 can be incorporated into the top corner of a door 603, which can have a transparent or translucent portion 624. Inside the door, a small pump 614 can be secured. If the door is at least partially transparent, the pump can be seen through the door 603. A door lock 623 can secure the door 603 in place and can help provide security for a medication bag 610 as well as the pump 614. An infusion line 608 can pass through a gap in an outer cover of the system 602 such as the door 603, allowing a pumping apparatus or structure in the pump 614 to interact with the tube or a volume connected therewith to urge fluid to flow from the medication bag 610 toward a patient or other fluid destination. This configuration can advantageously present a user with information regarding the contents of the system 602. For example, the current fill status of a medication bag 610 can be readily viewed, but not too easily accessed. If a pump 614 has an interface screen, it can convey information to a user despite being securely contained within the system 602 behind or within the door 603. In some embodiments, a layer over the pump can allow pump access (e.g., through a secure but pliable or heat-transmissible membrane or other layer). In this and other embodiments, the pump 614 can interact with the touch screen display 616. In this and other embodiments, the illustrated touch screen can be replaced or enhanced with a combination screen and soft/hard buttons. For example, the pump 614 can be a portable or ambulatory pump that concedes control of its pumping structures to the larger system 602 when it is plugged in or locked into the system 602. Electronic leads can facilitate this assumption of control, or wireless protocols can be employed, such as near field communication (NFC). Bluetooth. Wi-Fi, etc. If a touch screen display 616 is larger than a display of a pump 614, as shown here, or if a processor contained within a system 602 has greater capacity or speed than a processor contained within a pump 614, this can facilitate additional functions in the system 602 that may not be available in the pump 614 when it is being used in ambulatory or portable mode.

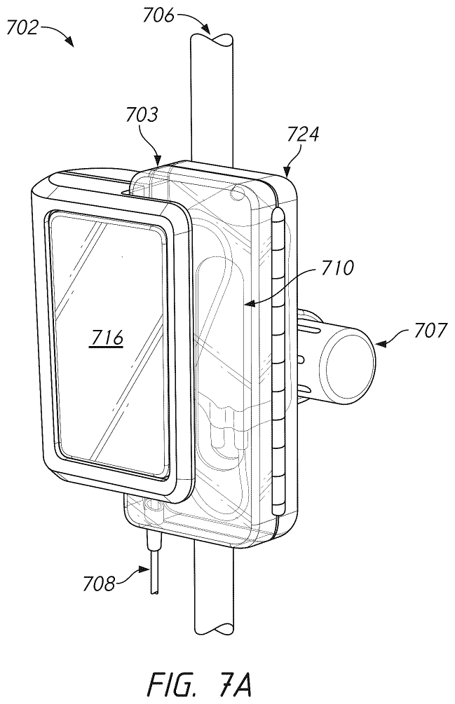

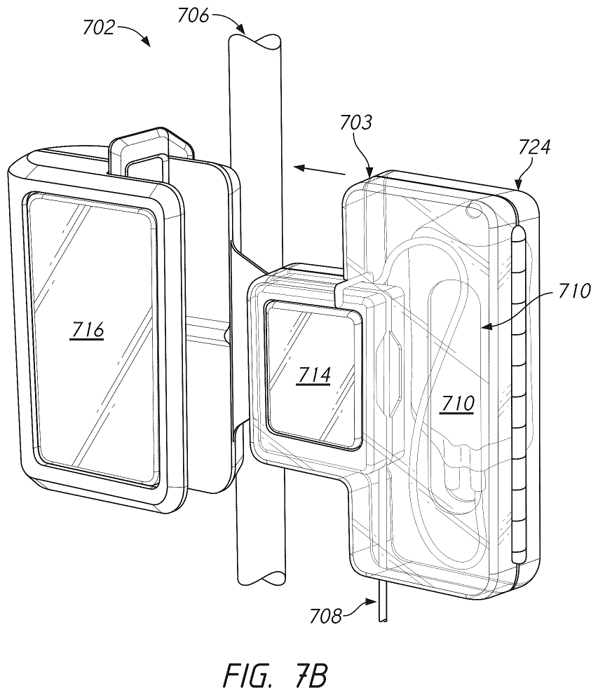

FIG. 7A shows a system 702 having a relatively large touch screen display 716 that wraps around or partially encloses a container 724. This is an example of embodiments configured to optimize or enhance touchscreen size and bag visibility. The container 724 can be transparent or translucent, and the system 702 including display 716 and container 724 (and embedded pump, not visible in FIG. 7A, but shown as 714 in FIG. 76 explained below) can be secured to an IV pole 706. For this purpose, a dial 707 can interact with a clamping mechanism that squeezes the pole 706, causing it to squeeze more or less tightly as the dial 707 is turned clockwise or counterclockwise. The container 724 can have a door 703, and a transparent approach can allow a fluid bag 710 to be seen such that its fill status is known to a medical provider or other user. A fluid line 708 can be seen exiting the container 724 at the bottom of this schematic diagram. The fluid line 708 is in fluid communication with the fluid bag 710 via the pump (shown as 714 in FIG. 7B) and can lead to a patient who receives the fluid for therapeutic purposes, for example.

FIG. 7B shows a configuration consistent with that of FIG. 7A. This shows how a large screen 716 can be incorporated into a partial enclosure that is configured to receive a pump 714, which can in turn be enclosed within the container 724. For this purpose, the container 724 can have a portion designed to receive the pump. This portion can have connections built in that interface with cooperating connections in the pump 714. These can be designed to facilitate data transfer, pump control, battery recharging, and cooling, for example, Dual metal leads can be used as positive and negative electrical charging connections. A large electrical contact can drain heat from a computer processor. Processors contained behind the display 716 and within the pump 714 can act cooperatively to increase overall processing power and functionality. Positioning a pump 714 directly behind a screen 716 can allow electrical connections to be simplified and facilitate contact-less charging and/or communication. Capacitive interactions can be used for devices in close proximity, which is possible with this approach. The slide-in configuration shown here can facilitate dual security features. For example, a lock located toward the leftward protruding portion containing the pump 714 can be obscured and less accessible to tampering when the container 724 is slid behind the screen 716.

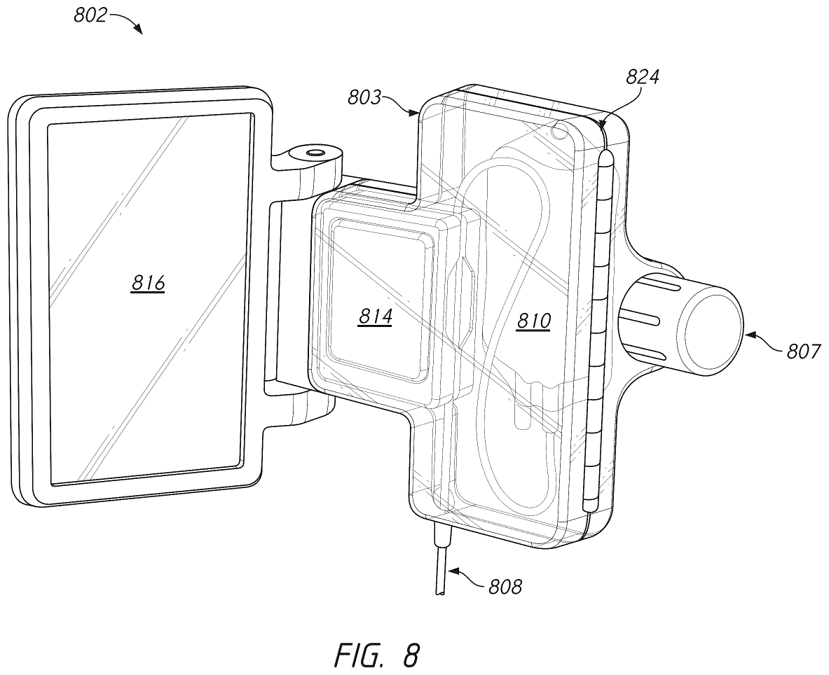

FIG. 8 shows a configuration consistent with many of the features and advantages in FIGS. 7A and 7B. Here, a pump can be mounted within an advanced lockbox. This configuration shows a display 816 that connects with a hinge and pivots with respect to a container 824. This configuration allows a side-by-side view of the display 16 and the contents of a container 824, which may be visible through an at least partially transparent door 803. An infusion line 808 can be seen exiting the container 824 and interacting with a pump 814 and a fluid (e.g., medication) bag 810. A transparent arrangement can allow visual verification of such fluid connections. A hinge at the right side of the container 824 can facilitate insertion and extraction of the contents of the container 924. The hinge connecting the display 816 can also allow a user to angle it quickly toward a viewing area, avoid glare, etc., without moving an IV pole, a clamp or clamp dial 807, or other mounting structure, for example. This configuration can hinge a screen around from the back, thereby allowing compact storage and movement, as well as full view of bag and pump, as well as a large screen or other interface, in a pole-mounted configuration.

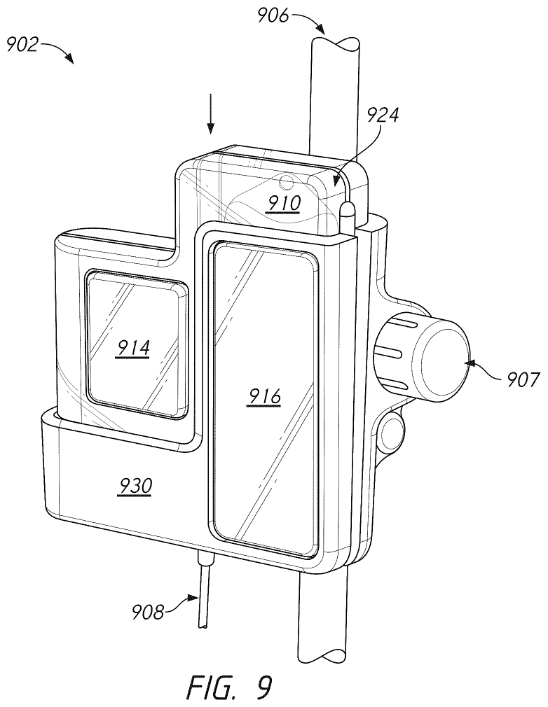

FIG. 9 shows a system 902 having a large screen interface 916 on a receptable structure 930 that can resemble a rigid pocket or caddy. This receptacle can be configured to contain a nested enclosure 924 that may incorporate or otherwise contain a pump 914. This can facilitate electrical connections between nested structure 924 and containing structure 924, as gravity helps position a pump 914 on a plug or other interface within the receptable 930. If the nested structure 924 has a transparent aspect, a fluid receptacle 910 (such as an IV bag) can be viewed and its status evaluated from outside. If the nested structure 924 has a transparent aspect, a fluid receptacle 910 (such as an IV bag) can be viewed and its status evaluated from outside. Alternatively, the front of the shown system could open up in the same orientation as shown in FIG. 8B, with the screen interface 916 and receptacle 930 opening to reveal the fluid receptacle 910 and pump 914. As with other described configurations, a dial 907 can interact with a clamp to secure the system 902 to a pole 906.

### Advantages

Hybrid systems, pumps, and advanced lockboxes as described herein can enable broad application, without accommodations, of one pump across the continuum of care through leverage of an advanced accessory. A combined hybrid system can complement full-service bedside pump systems to complete or enhance a pump portfolio with just one incremental pump, for example, A “best of both worlds” approach such as described here can support a small, inexpensive, and simplified ambulatory pump, with acute care pain management needs realized through the system (pump+advanced lock box). The described systems can support alignment to an ambulatory pump to support acute care PCA, providing reach into neuraxial (e.g., epidural and intrathecal modes) as well as home infusion opportunities. In some countries, patient-controlled analgesic (PCA) systems may not present great market demand. Nevertheless, they often are a necessary product. The hybrid approaches described herein allow an ambulatory pump to “look and feel” like part of another (potentially more sophisticated or otherwise different) pump or product family.

The present disclosure addresses limitations of smaller ambulatory pumps (e.g., those having a small user interface due to overall small size of the pump). Once a small ambulatory pump is mounted within a secure lock box that holds a fluid bag, the overall system mounted to a pole in an acute care setting can be large. By introducing a large touch screen to the lock box, a reasonably sized system can optimize the user interface size (as disclosed herein), which improves ease of programing and infusion review, and also supports assessment of the pump from a further distance, such as a hospital room doorway.

The present disclosure provides many additional advantages. Described embodiments can optimize ambulatory and pole-mounted PCA products from a single pump. Broad clinical applications of a single ambulatory pump can be achieved through the application of complementary accessories. A very small ambulatory pump can meet alternate site customer preferences but can still be clinically relevant in the hospital setting. Disclosed embodiments streamline product use and feel by using concurrent design and optimization of pump and accessories. These embodiments can drive efficiencies in product development and in the market by enabling broad clinical applicability of one pump, transferring significant “look and feel” requirements to an accessory. Disclosed embodiments can transfer significant features from a pump to an accessory, enabling a very low cost ambulatory pump that can deliver advanced features to the appropriate markets through an integrated accessory.

### Variations and Features

There are multiple variations and features consistent with the present disclosure. For example, a pump may still be operated completely independently of an integrated accessory (e.g., a smart lockbox and recharging station), which is to say the pump has a high level of functionality on its own. Incremental functionality may be available only when a pump is used in conjunction with an advanced accessory. The pump may still be operated with other more traditional “single purpose” accessories.

### Accessory Features

An accessory holds the pump and may do one or more of the following. It may support multiple channel operation via mounting and integration of multiple pumps. The pump screen and controls may or may not be fully or partially visible and/or accessible to the caregiver. For example, in some embodiments, a user may need to be able to see and access a stop button. The pump itself may or may not be seen when interfaced to the accessory. The pump may or may not be separated from the accessory during operation under certain therapies (without losing infusion continuity). The pump(s) may mount into or onto the accessory in a manner which makes the pump easy to disengage, but only when there is not a therapy underway. The pump(s) may mount into or onto the accessory in a manner which allows the pump to be disengaged only through use of a security device or procedure (e.g., code, key, etc.) to unlock the box, for example. The accessory can comprise a stationary docking station, which can have a large user interface, for example, (Stationary may refer to a larger device that is mounted on a wheeled pole or other holder but is nevertheless less portable than another related device). The accessory can hold or support or secure a smaller or modular and portable pump device. The pump device can be removed or disengaged from the accessory in a non-technical manner, such as without removing a housing, without tools and/or by a person without technical training. For example, in some embodiments and/or modes, rapid, non-technical disengagement can comprise a hospital user rapidly and conveniently detaching the pump manually (e.g., by unsnapping, gently lifting, unlatching, using a simple button or other actuator, opening a lid, etc.). Authorization for pump detachment or disengagement can be strictly enforced (through biometric or other locks, etc.), while still making the physical act of detachment relatively rapid and straightforward, once authorized (e.g., once a lock is released).

The accessory may include any or all of the following features, in any combination: physical and electronic interfaces to enable mounting of the pump and transfer of the AC power, patient bolus cord, communication interface(s) electrical access and control functions from the pump connectors to the accessory or accessory connectors (for example, the bolus cord connector on the pump is now unavailable and/or disabled and a bolus cord connector in the accessory becomes the active port). The “connection” between the pump and accessory can be through electrical connectors on both or can be via remote communications between the two, such as Bluetooth. The accessory may include: wired interfaces such as USB ports; a locking box to secure and envelope the pump and infusion bag and element of the infusion set directly mating with the pump;

In preferred embodiments, the locking box enables only authorized users to have medication access by unlocking the pump's locking box by using a single or configurable combination of locking mechanisms, such as but not limited to the scanning the authorized clinician's healthcare provider ID (e.g., barcode. RFID and etc.), entry of a healthcare provider defined passcode on the electronic supplemental screen/user interface of the accessory or a high security lock and key on the locking box. In addition, the pump or system advantageously can detect whenever the locking box is closed and locked. In some embodiments, the pump provides an audible and visual alarm if the locking box has been forcefully opened. The locking box may be illuminated using an illumination color that defines the infusion delivery route of the medication (e.g., yellow to represent epidural, etc.) or medication class (e.g., opioid, local anesthetic, etc.).

In some embodiments, the accessory can hold and mechanically secure medications in a bottle, vial, or flexible container.

The accessory can include a pole clamp. This can be locking or non-locking (locks the accessory to a pole or not). The pole clamp can be detached from the accessory, or, in some embodiments, is permanently attached. The locking pole clamp accessory may use a high security lock and key and/or electronic locking mechanism. In some embodiments, the pump can detect when the pole clamp is secured and locked onto the IV Pole by providing a notification (e.g., visual and audible).

The accessory can include a screen or display. This may further be a touch screen, and it may complement an on-pump screen/user interface content or may replace it with either larger version of the pump interface or provide a modified screen layout or content versus what the on-pump screen would otherwise provide. The screen may include content that would not be available on the pump. Some embodiments use a display color and text to define the infusion delivery route of the medication (e.g., epidural, etc.) or medication class (e.g., opioid, local anesthetic, etc.).

The accessory can include an AC power adaptor and wall cord. This may satisfy one of numerous standards, as appropriate per market (blade configurations, power rating). This may also be permanently affixed or may be dis-connectable.

The accessory can include a rechargeable battery, a carrying strap, a handle, and or a bar-code or RF reading capability to confirm the medication stored in the accessory. This can be integrated such that it identifies the medication and eliminates the ability to scan one medication and utilize another. This can alternatively be more external in design, where a handheld or wand is used further for the identification of the patient, caregiver, etc.

The accessory can include pump user interface controls, including on-off buttons, an emergency pump pause/stop button, wireless communication capabilities (e.g., cellular, WiFi, Satellite), and/or antenna(s). It can be configured to recharge the pump if the pump has an internal or primary rechargeable battery/ies. The accessory can allow for troubleshooting the pump and or run diagnostics. In some embodiments, the accessory contains biomedical protocols and any interfaces required.

The accessory can include electronic capacity such as: a memory (for example, transfers the pump history from the device to the accessory for upload to a server or networked safety software platform); one or more a processors; signal conditioning and amplification (including, for example, sensors). The accessory may replace the operation of the pump electronics or may complement it.

The accessory can include safety software capacity. For example, it may make HMMS (Hospira Medication Management Software, such as MedNet or LifeShield) available when the pump operates in conjunction with the accessory, but can use an alternate safety software or minimized HMMS when the pump operates independently. In some embodiments, when the pump runs independently the drug library is available and active on the pump but when the pump is interfaced with the accessory full, persistent connectivity between pump and HMMS is added or enabled.

The accessory can include language translation capabilities. For example, the ambulatory pump may provide one or a few languages but the accessory can enable operation in incremental languages, either through configuration or, for example, through point-of-care arrival of a nurse with a badge that shows she prefers to program the pump in an alternate language.

The accessory can include: fluid or humidity sensing to quickly identify a bag, set or connection leak within the accessory housing; still or video camera capabilities; mass or fluid sensing capability to provide the remaining volume in a bag (particularly in embodiments where the medication reservoir is not fully or partially viewable); temperature sensing; the ability to accept input from physiological sensors such as temperature, respiration rate, heart rate. SpO2, etCO2, etc. (a dedicated circuit board to support such sensing can be housed in the advanced locking box). The accessory can further present data from other sensors such as infiltration detection, patient vital signs, physiological sensors, etc. A dedicated circuit board to support such sensing can be housed in the advanced locking box.

In some embodiments, the accessory can: present data from other sources (“data portal” type applications, e.g., a source such as Theradoc, Capsule, Galileo, etc.—companies or systems that consolidate patient data from numerous sources and makes it available via personal computer, a PCA pump, or other device); control or stop pump operation based on certain sensor data (for example, cease PCA delivery based on respiratory depression); and/or confirm the chemical composition of the fluid flowing from bag to patient. Having a large screen (e.g., touchscreen) can aid in these data presentation and control functions.

An infusion bag or other container may be partially or completely viewable through a clear front door or side surface or window or may not be viewable, with an internal camera providing remaining volume. And the accessory may include an automated patient blood sampling and analysis system (coagulation, blood sugar, electrolytes, etc.)

Some of the above therapeutic capabilities can be made available only when a pump is connected to the accessory. The accessory and the associated pumps can have pre-defined product lifetimes. Some of the above features can be on the pump itself, on the accessory, or both.

The pump can directly interface with the accessory as outlined above, or a partial pump version can integrate with the accessory (some of its external parts removed or never included in manufacturing). A pump that interfaces with the accessory in a more permanent fashion may not require a display, user entry buttons, complete outer housing, etc. So the integrated accessory/pump combo can comprise a comprehensive product, modular only in a manufacturing sense (as an alternative to embodiments discussed above that are modular at a point-of-use sense).

### Disclosed Embodiments

Embodiments demonstrating the features and advantages described herein can be described as follows. A portable infusion pump that can be operated in two modes: portable mode, wherein it operates independently to provide full user control and access; and secured mode, wherein it fits within and cooperates with a pump controller/container.

A multi-function, modular pump system can have a portable infusion pump configured for ambulatory use. The portable infusion pump can have a rechargeable battery, a portable user interface, and a controller/container. In some embodiments, the portable user interface can be significantly smaller than the stationary user interface on the stationary docking station to help make the portable infusion pump smaller, lighter, and less bulky. The controller/container can have a security feature configured to prevent removal or unauthorized access and a pump mount configured to engage and stabilize the pump within the container, allow pump to maintain infusion continuity when removed, and enable rapid disengagement when authorized. An external user interface can allow for control of the contained pump, review of pump run status, log event history, settings, and configurations. A machine interface can allow control signals to pass from external interface to a contained, engaged portable pump. A power module can be configured to provide power to both the contained portable infusion pump and the pump controller/container. A simplified extending patient control can be configured to provide a bolus dose. The portable pump can be configurable for use in two modes: portable mode, and secured mode.

### TERMINOLOGY AND CONCLUSION

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the an from this disclosure, in one or more embodiments.

As used in this application, the terms “comprising,” “including,” “having,” and the like are synonymous and are used inclusively, in an open-ended fashion. and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

Embodiments of the disclosed systems and methods may be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Per, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules, but may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

A number of applications, publications, and external documents may be incorporated by reference herein. Any conflict or contradiction between a statement in the body text of this specification and a statement in any of the incorporated documents is to be resolved in favor of the statement in the body text.

Terms of equality and inequality (less than, greater than) are used herein as commonly used in the art, e.g., accounting for uncertainties present in measurement and control systems. Thus, such terms can be read as approximately equal, approximate less than, and/or approximately greater than. In other aspects of the invention, an acceptable threshold of deviation or hysteresis can be established by the pump manufacturer, the editor of the drug library, or the user of a pump.

While the embodiments of the invention disclosed herein are presently considered to be preferred, various changes and modifications can be made without departing from the scope of the invention. Although described in the illustrative context of certain preferred embodiments and examples, it will be understood by those skilled in the art that the disclosure extends beyond the specifically described embodiments to other alternative embodiments and or uses and obvious modifications and equivalents. Thus, it is intended that the scope of the claims which follow should not be limited by the particular embodiments described above. The scope of the invention is indicated in the appended claims, and all changes that come within the meaning and range of equivalents are intended to be embraced therein.

The following is claimed:

1. A multi-function, modular pump system comprising:
a stationary docking station comprising:
at least one security feature configured to prevent unauthorized removal of or access to at least one of the docking station, a portable pump attachable to the docking station, and a medication reservoir container configurable to be in fluid communication with the pump;
a pump mount configured to engage and stabilize the pump within the docking station, allow the pump to maintain infusion continuity when removed, and enable rapid, non-technical disengagement of the pump from the docking station when authorized; and
a stationary external user interface configured to allow user control of the pump when the pump is attached to the docking station and review of pump run status, event history, and settings;
the portable pump comprising an infusion pump configurable to be separable from the docking station and, when separated, function independently for ambulatory use, the portable pump further comprising:
a battery and a power input structure connected to the battery;
a portable user interface; and
the medication reservoir container.

2. The system of claim 1, wherein the stationary docking station further comprises a machine interface configured to allow control signals and other information to pass between the external user interface and the contained, engaged portable pump.

3. The system of claim 2, wherein the stationary docking station further comprises a power module configured to provide power to both the contained portable pump and the stationary docking station.

4. The system of claim 2, wherein the external user interface comprises a large touch-screen.

5. The system of claim 3, wherein the battery of the portable infusion pump is rechargeable and the power input structure is configured to interface with the power module and a charging structure in the stationary docking station to recharge the battery.

6. The system of claim 1, further comprising a simplified extending patient control configured to provide a bolus dose.

7. The system of claim 1, wherein the portable infusion pump is configurable for use in portable mode and secured mode.

8. The system of claim 7, wherein in portable mode, the pump operates independently of the stationary docking station.

9. The system of claim 7, wherein in secured mode, the pump fits within and cooperates with the stationary docking station, and the system supports additional functionality comprising one or more of stronger communications hardware and sensor support.

10. The system of claim 9, wherein stronger communications hardware comprises Wi-Fi communications or cellular communications, and sensor support comprises interfaces and support monitoring information including one or more of SpO2, EtCO2, minute ventilation, and patient vitals.

11. The system of claim 9, wherein sensor support is configured to monitor for opioid induced respiratory depression.

12. A modular pump system comprising:
a portable infusion pump configurable for independent operation in a first mode and having a medication container incorporated therewith; and
a pump docking module configured to securely house the portable infusion pump and medication container, the docking module comprising a user interface and configured to operatively and persistently communicate with the portable infusion pump while the pump is housed therein.

13. The system of claim 12, wherein the pump docking module is configured to engage and stabilize the pump therein allowing pump to maintain infusion continuity when removed, and enable rapid disengagement when authorized.

14. The system of claim 12, the pump docking module user interface comprising a color touch screen that provides interactive control and display responsibilities of the portable infusion pump when that pump is mounted within the docking module.

15. The system of claim 12, wherein the docking module user interface is configured to assume interactive control and display responsibilities of the portable infusion pump when that pump is mounted within the docking module.

16. The system of claim 12, wherein the docking module locks to secure the pump and medication within the module and can be unlocked by one or more of: a physical key, a passcode entry, and a clinician badge.

17. The system of claim 12, wherein the docking module is secured to IV pole such that it is releasable only by one or more of a physical key, a passcode entry, and a clinician badge.

18. The system of claim 12, wherein a power submodule within the docking module is configured to convert AC power, power the docking module features, and power and recharge a battery of the portable infusion pump.

19. The system of claim 12, further comprising a patient bolus cord configured to communicate to the pump directly or through the docking module.

20. The system of claim 19, wherein a patient bolus control wirelessly communicates to the pump and includes a battery rechargeable by the docking module.

21. The system of claim 12, wherein the docking module interface is configured to obtain and display the pump infusion history, including total medication delivered over time, timing of patient bolus requests and delivered patient boluses.

22. The system of claim 12, wherein the pump and docking module, when integrated, communicate to each other through a wired connection upon pump mounting into the controller/container.

23. The system of claim 12, wherein the pump and docking module, when integrated, communicate to each other through a wireless connection upon pump mounting into the controller/container.

24. The system of claim 12, wherein the docking module includes a communication means configured to communicate from the system to a networked electronic health system.

25. The system of claim 12, wherein the docking module is configured to hold and secure medication presented in a bag, semi-rigid container or syringe.

26. The system of claim 12, wherein the docking module includes electronics and software to interface to external sensors comprising at least one of SpO2, EtCO2, minute ventilation and patient vital signs sensors, and wherein external sensor output is available through an externally-accessible interface of the docking module.

27. The system of claim 26, wherein external sensor output is digested by an algorithm operable in the system to notify the pump user and/or automatically stop the pump under defined conditions of respiratory depression.

28. The system of claim 12, further comprising a database and at least one signal associating within the system the pump, a particular user of the pump, that user's medication, and the medication order.

29. The system of claim 28, further comprising a bar code scanner or near field sensing system.

30. The system of claim 12, further comprising a processor, mode algorithm, and mode indicator, collectively configured to modify a mode indicator to inform a user of the present mode, the modes linked to at least route of delivery or family of medication.

31. The system of claim 29, wherein the docking module is further configured to indicate a current delivery mode using color coding.

32. The system of claim 29, wherein an outer surface of one or more of the portable infusion pump and the pump docking module is configured to indicate a current delivery mode using color coding.

33. The system of claim 12, wherein the pump docking module is configured to securely house the portable infusion pump by electronically recognizing and logging at least one of the following actions for a door of the pump docking module: locking, opening, closing, and unlocking.

34. The system of claim 12, wherein the pump docking module is configured to securely house the portable infusion pump by electronically recognizing and logging at least one of the following actions: locking, attaching, detaching, and unlocking of the pump docking module to and from a pole.
